(19) United States
(12) Patent Application Publication
(10) Pub. No.: US 2010/0012859 A1
(43) Pub. Date: Jan. 21, 2010

(54) Method For Treating A Target Volume With A Particle Beam And Device Implementing Same

(76) Inventor: Yves Claereboudt, Corbais (BE)

Correspondence Address: FITCH EVEN TABIN & FLANNERY, 120 SOUTH LASALLE STREET, SUITE 1600, CHICAGO, IL, 60603-3406, US

(21) Appl. No.: 12/307,210

(22) PCT Filed: Mar. 2, 2007
(86) PCT No.: PCT/EP07/52021

§ 371 Date: May 29, 2009

(30) Foreign Application Priority Data
Jul. 6, 2006 (EP) 06116756.5

(51) Int. Cl. A61N 5/10

(52) U.S. Cl. 250/492.3

(57) ABSTRACT

A method for treating or irradiating a target volume (10) with a charged particle beam (100), to the target volume being associated three coordinates according to a X, Y and Z direction, the Z coordinate corresponding to the beam direction while the X and Y coordinates correspond to directions perpendicular to the Z direction, said charged particle beam producing in said target volume an irradiation spot (101), the method comprising the following steps:—continuously scanning said spot in the X and Y directions and applying a continuous movement to said spot in the Z direction by seamlessly modifying the energy of said beam, the step of applying a continuous movement in Z direction and the step of continuous scanning in X and Y direction being performed simultaneously, thereby performing a continuous 3D scanning of the target volume.

### FIELD OF THE INVENTION

The present invention relates to a method for treating a target volume with a charged particle beam, in particular a proton or light ion beam.

The present invention also relates to a device for carrying out said method.

The field of application is the proton or light ion therapy used in particular for the treatment of cancer, in which it is necessary to provide a method and device for irradiating a target volume constituting a phantom for delivery tests or a tumour to be treated.

### STATE OF THE ART

Radiotherapy is one of the possible ways for treating cancer. It is based on irradiating the patient, more particularly his or her tumour, with ionizing radiation. In the particular case of proton therapy, the radiation is performed using a proton beam. It is the dose of radiation thus delivered to the tumour which is responsible for its destruction.

In this context, it is important for the prescribed dose to be effectively delivered within the target volume defined by the radiotherapist, while at the same time sparing as much as possible the neighbouring healthy tissues and vital organs. This is referred to as the “conformation” of the dose delivered to the target volume. In order to reach a suitable conformation, a predefined radiation dose is calculated in order to reach a clinically useful dose distribution that conforms, as far as possible, the shape of the target volume and contemporaneously spares the contiguous healthy tissues. Various methods which may be used for this purpose are known in proton or light ion beam therapy, and are grouped in two categories: “passive” methods and “active” methods.

Whether they are active or passive, these methods have the common aim of manipulating a proton or light ion beam produced by a particle accelerator so as to completely cover the target volume in the three dimensions: the “depth” (in the direction of the beam, hereafter called “Z direction”) and, for each depth, the two dimensions defining the plane perpendicular to the beam. In the first case, this will be referred to as “modulation” of the depth, or alternatively modulation of the path of the protons into the matter, whereas, in the second case, this will be referred to as the shaping of the irradiation field in the plane perpendicular to the beam.

Passive methods which represent traditional beam delivery techniques use an energy degrader to adjust the path of the protons to their maximum value, corresponding to the deepest point in the area to be irradiated, using a rotating wheel of variable thickness to achieve modulation of the path (the latter device thus being referred to as path modulator). The combination of these elements with a “path compensator” (or “bolus”) and a specific collimator makes it possible to obtain a dose distribution which conforms closely to the distal part of the target volume. However, a major drawback of this method lies in the fact that the healthy tissues located upstream and outside of the target volume are themselves also occasionally subjected to large doses. Furthermore, the need to use a compensator and a collimator which is specific to the patient and to the irradiation angle makes the procedure cumbersome and increases its cost.

Among active methods, the pencil beam scanning is a very well known scanning method, wherein the movement of the particle beam is performed in two directions perpendicular to the direction of the beam defining the irradiation plane. The intersection of the beam with said irradiation plane is representing the spot of irradiation.

More particularly, the movement in the two directions perpendicular to the direction of the beam takes place with the help of electromagnets controlling the position of the beam and in particular of the spot. This is performed by applying a current of a known magnitude to said electromagnets thereby generating a magnetic field of predictable intensity which allows the bending or deflecting of the beam (depending on the magnetic rigidity of the particles of the beam).

Preferably, the scanning with the help of said electromagnets takes place in such a way that a continuous movement of the spot is applied in the (X, Y) plane perpendicular to the beam direction (Z). The (X, Y) plane is called the irradiation plane.

It is thus observed that the conformation to the target volume is achieved without the use of variable collimators and solely by an optimal control of the path of movement of said spot. The target volume is cut into several successive planes perpendicular to the direction of the beam, corresponding to successive depths, the depthwise movement of the spot from one plane to another being achieved by modifying the energy of the particle beam.

The movement of the spot from one irradiation plane to another is effected by modifying the energy of the particle beam using e.g. an energy degrader, as in patent document U.S. Pat. No. 6,433,336. It should be noted that the movements from one irradiation plane to another irradiation plane are considered to be non-continuous.

Advantageously, the energy of the particle beam is modified immediately after it is extracted from the accelerator.

According to U.S. Pat. No. 6,717,162 B1, an improvement has been suggested, whereby the variation of the intensity of the particle beam and the movement of said spot along the two directions perpendicular to the particle beam is performed simultaneously in order to obtain a better conformation of the dose delivered to the target volume.

However, though it is possible to modify the energy of the beam without interrupting said beam irradiation, the movement of the spot along the Z direction still seems to be non continuous.

All these techniques proceed in a similar way to deliver the dose to a target volume as follows:
Producing a beam with a defined energy in order to reach a certain depth in the target volume along the axis Z;
Delivering the produced beam along two axes perpendicular to the Z-axis in order to reach a desired dose within the target area. The delivery of this mono-energetic beam is called irradiating a layer;
Producing another beam with another energy in order to reach another depth in the target volume along the axis Z;
Repeating the previous steps in order to irradiate the entire target volume of the tumour.

Nevertheless, both active and passive techniques have several drawbacks:
the conformity of the delivered dose to the desired dose is sensitive to the relative precision in the energy of the beam produced for each layer;
the conformity of the delivered dose to the desired dose is sensitive to small motions of the target volume that could occur in between two layers (e.g. in case of living tissues);
the production or preparation of a beam of another energy takes time, which can be considered as an undesired overhead that slows down the irradiation method;
for the discretisation along the third (Z) axis (through beam energy), a compromise needs to be found between the number of layers which impacts the time needed to irradiate the target volume and the non-conformities appearing in the dose distribution along this third axis;

depending on the number of layers, one observes a “wave” of the dose around the desired dose, in the Z direction, corresponding to the superposition of individual Bragg peaks;
some charged particles such as light ions or proton of low energy generate such thin dose deposition along the third axis that either the number of layers need to be increased significantly or additional devices (such as a ridge filter) need to be used to artificially extend the dose distribution along this axis. These devices however deteriorate the characteristics of the beam and their use should be avoided when possible.

### AIMS OF THE INVENTION

The present invention aims to provide a method, a software and a device for treating a target volume with a particle beam, which avoid the drawbacks of the methods described previously, while at the same time making it possible to deliver a dose to the target volume with the greatest possible conformity and/or flexibility.

The present invention aims in particular to provide a method, a software and a device which dispense with a large number of auxiliary elements such as collimators, compensators, diffusers or even path modulators.

The present invention aims also to provide a method, a software and a device which makes the irradiation more insensitive to organ motion along the axis of irradiation.

The present invention aims also to provide a method, a software and a device which make it possible to obtain protection against an absence of emission of the beam (blank or hole) or against an interruption of the movement of said beam.

In particular, the present invention aims to provide a method, a software and a device which make it possible to obtain a ratio of highest to lowest dose in the target volume ranging from 1 to 500.

### SUMMARY OF THE INVENTION

The present invention is related to a method, a software and a device for irradiating a target volume with a charged particle beam, as set out in the appended claims, wherein the target volume is scanned continuously along the three directions of space by said charged particle beam. The method, the software implementation and the device of the invention are arranged to be manipulated by a physicist or a mathematician. They are not intended to be manipulated by general clinicians.

According to a first aspect of the invention, there is provided a method for treating or irradiating a target volume with a charged particle beam. To the target volume are associated three coordinates according to a X, Y and Z direction, the Z coordinate corresponding to the beam direction while the X and Y coordinates correspond to directions in a plane that is perpendicular to the Z direction. The charged particle beam produces in said target volume an irradiation spot. The method comprises the following steps:
continuously scanning said spot in the X and Y directions and
applying a continuous movement to said spot in the Z direction by seamlessly modifying the energy of said beam, the step of applying a continuous movement in Z direction and the step of continuous scanning in X and Y direction being performed simultaneously, thereby performing a continuous 3D scanning of the target volume.

Preferably, the method for treating or irradiating a target volume according to the invention comprises the steps of:
measuring and checking continuously the position of the spot in the target volume;
adapting and correcting the position of said spot through a control loop for real time correction.

More preferably, the method of the invention comprises the step of modifying the scanning speed of the spot according to the 3D directions (X, Y, Z) while the target volume is being irradiated. Preferably, the method of the invention comprises the step of modifying the beam intensity while the target volume is being irradiated.

Preferably, in the method of the invention, the 3D scanning and the modification of the beam intensity are performed simultaneously. More preferably, the abovementioned modifications are (pre-)determined by an algorithm for planning.

According to a second aspect of the invention, there is provided a device for irradiating a target volume with a charged particle beam, wherein said charged particle beam generates an irradiating spot located within the target volume and to which are associated three coordinates according to a X, Y and Z direction, the Z coordinate corresponding to the beam direction while the X and Y coordinates correspond to directions perpendicular to the Z direction. The device of the invention comprises:
scanning means for deflecting the particle beam along the X and Y directions,
energy variation means arranged for continuously varying the energy of the beam and
central controlling means adapted to control continuously the scanning means and the energy variation means by a planning and control algorithm in order to obtain a conformation of the irradiation dose to the target volume.
   The device of the invention preferably comprises all the adequate means to put the method of the invention into practice.

Preferably, the scanning means of the device according to the invention are arranged for moving the spot continuously in both X and Y directions. More preferably, the energy variation means of the device of the invention are arranged for seamlessly varying the energy of the beam, thereby moving the spot continuously in Z direction.

Preferably, the central controlling means of the device of the invention allow a volume element within the target volume to be irradiated several times by the spot.

Preferably, the device of the invention further comprises a particle accelerator arranged for producing the charged particle beam. More preferably, the energy variation means for the particle beam are located immediately after the extraction of the charged particle beam from the accelerator. Even more preferably, the energy variation means for the particle beam comprise an energy degrader, an energy absorber, or an energy selection device.

Preferably, the device of the invention comprises an irradiation head. The scanning means preferably comprise a scanning magnet for each of the X and Y directions. The scanning magnets are preferably located in the irradiation head.

Preferably, the device of the invention comprises beam intensity variation means arranged for seamlessly varying the intensity of the charged particle beam.

Preferably, the central controlling means of the device of the invention are adapted to carry out the control of the scanning means, of the energy variation means and possibly of the beam intensity variation means with the help of a planning and control algorithm. The algorithm preferably makes use of a control loop correcting in real time the trajectories of the spot.

Preferably, the device of the invention comprises a computer system or a sequencer implementing a treatment algorithm allowing the determination of an irradiation dose corresponding to each irradiation volume or voxel by predetermining the beam intensity, the beam energy and the scan speed in the X, Y and Z directions for each irradiation volume or voxel.

Preferably, the scanning means, the energy variation means and possibly the beam intensity variation means of the device of the invention act by moving the spot with no interruption of the beam.

Preferably, the device of the invention comprises at least one detection device such as an ionization chamber and/or a diagnostic element, allowing measurements to be performed so as to check the conformation of the irradiation dose to the target volume.

According to a third aspect of the invention there is provided a software program for being run on a computer. The sequencer or the software program is arranged for generating controlling commands to the scanning means and to the energy variation means of the device of the invention, in order to obtain the continuous movement of an irradiation spot according to the method of the invention.

According to a fourth aspect of the invention there is provided a use of the method and/or the device of the invention in cancer therapy. The method and device of the invention hence may be used for the treatment of cancer.

### BRIEF DESCRIPTION OF THE FIGURES

All drawings are intended to illustrate some aspects and embodiments of the present invention. The drawings described are only schematic and are non-limiting.

FIG. 1 represents an exploded view of the device for allowing irradiation in order to treat a target volume according to the present invention.

FIGS. 2a and 2b are representing methods for scanning with a particle beam a target volume according to the state of the art and to the present invention respectively.

FIGS. 3 and 4 represent a 3D scanning of a target volume by applying the method according to the present invention.

### DESCRIPTION OF A PREFERRED EMBODIMENT OF THE PRESENT INVENTION

One or more embodiments of the present invention will now be described in detail with reference to the attached figures, the invention is not limited thereto but only by the claims. The drawings described are only schematic and are non-limiting. In the drawings, the size of some of the elements may be exaggerated and not drawn on scale for illustrative purposes. The dimensions and the relative dimensions do not necessarily correspond to actual reductions to practice of the invention. Those skilled in the art can recognize numerous variations and modifications of this invention that are encompassed by its scope. Accordingly, the description of preferred embodiments should not be deemed to limit the scope of the present invention.

Furthermore, the terms first, second and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. The terms so used are interchangeable under appropriate circumstances and the embodiments of the invention described herein can operate in other orientations than described or illustrated herein. For example “underneath” and “above” an element indicates being located at opposite sides of this element.

It is to be noticed that the term “comprising”, used in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

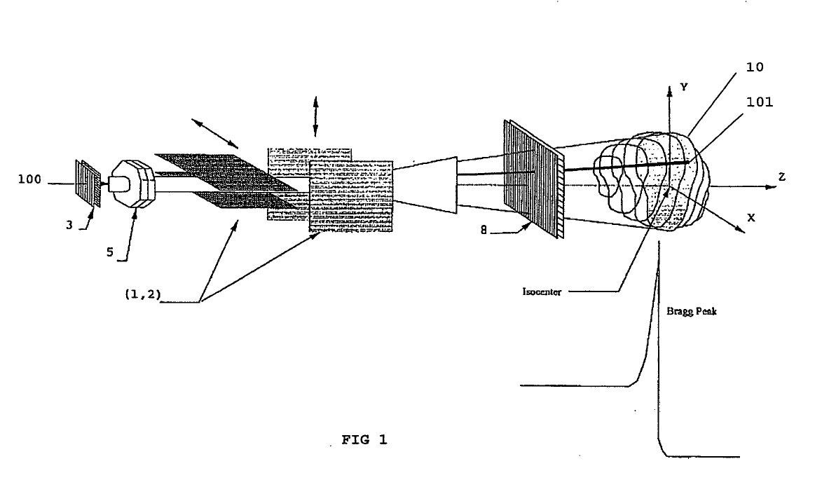

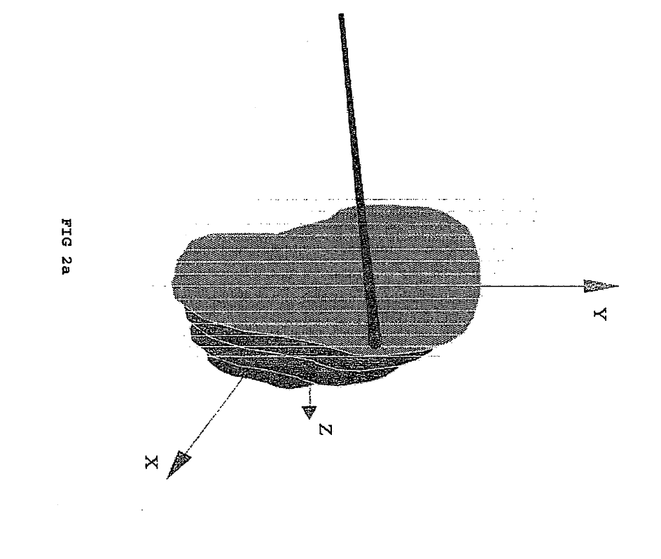

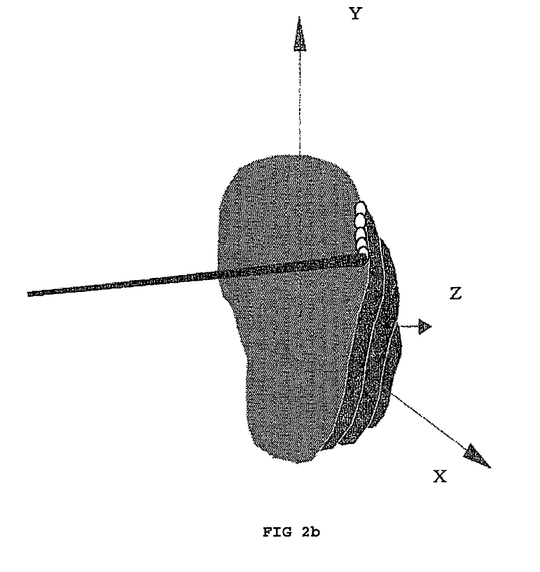

The present invention aims to provide a method and a device for treating a proton or light ion beam produced by an accelerator devoted to the irradiation of a target volume consisting, for example, of a tumour to be treated in the case of a cancer or a phantom for delivery tests, and which have improvements over the prior art described in FIG. 1.

To do this, it is intended to move a spot produced from this proton or light ion beam along the three dimensions directly in the patient's body in order to cover the target volume in the three dimensions.

FIG. 1 partially shows the device for carrying out the method according to the present invention. According to one preferred embodiment, a cyclotron (not shown) is used to produce a proton or light ion beam 100 generating a spot 101 to be moved. Means (not shown) are provided for modifying the energy of the proton or light ion beam immediately after it is extracted from the accelerator in order to allow the movement of the spot in the longitudinal dimension, that is to say in the direction of the beam, so as to define the Z coordinates.

The device for carrying out the above method requires at least the following elements:
means for the production of a charged particle beam, such as a cyclotron,
means for controlling the intensity of the produced beam, such as a controllable and regulated ion source,
means for applying a continuous movement to the beam in the X and Y directions perpendicular to the Z direction of the beam,
means for adapting seamlessly the energy of the particle beam such as a continuous absorber/degrader and/or an energy selection device and
central controlling means, such as a computer or computer network, for controlling the above cited means.

A continuous absorber can be made by providing a single or a double wedge, movable so as to vary the depth of material traversed by the beam. The material can be graphite or beryllium.

In order to modify the energy of the emitted beam, an energy degrader is preferably used, and more particularly a continuous energy degrader with characteristics similar to those disclosed by U.S. Pat. No. 6,433,336, having the same assignee, possibly modified with a modified wheel having smooth faces instead of staircase faces.

Advantageously, the designed device will start to produce a beam at the highest required energy in order to reach the deepest part of the target volume to be irradiated. The device will start the scanning along the X & Y axes while simultaneously, continuously and comparatively slowly change the energy of the incident beam and accordingly the Z-coordinate. The Z-direction corresponds to the direction that the beam assumes in un-scanned state (corresponding to the centre of the (X, Y) scanning area).

The present invention also aims to provide a method for treating or irradiating a target volume with a charged particle beam produced by an accelerator, said charged particle beam producing on said target volume an irradiation spot, comprising the following steps:
applying a continuous movement to said spot with the help of two scanning magnets in the (X, Y) directions perpendicular to the direction (Z) of the beam;
applying a continuous movement to said spot in the direction Z, by modifying the energy of said beam during the scanning of the beam in the (X, Y) directions perpendicular to the direction (Z) of the beam, thereby performing a continuous 3D scanning of the target volume.

In normal conditions, the irradiation of the whole target volume should not be paused and the scanning will be a continuous 3D scanning by
adapting the scanning pattern to the desired volume to be irradiated (X,Y,Z),
adapting the beam intensity and/or scanning speed (potentially stopping the scan) to obtain the desired dose deposition at the current location (X, Y & Z) of the spot pencil beam and
adapting the scanning pattern to the change of magnetic rigidity of the incident beam.
   All steps are performed in real time with the aid of a planning and control algorithm.

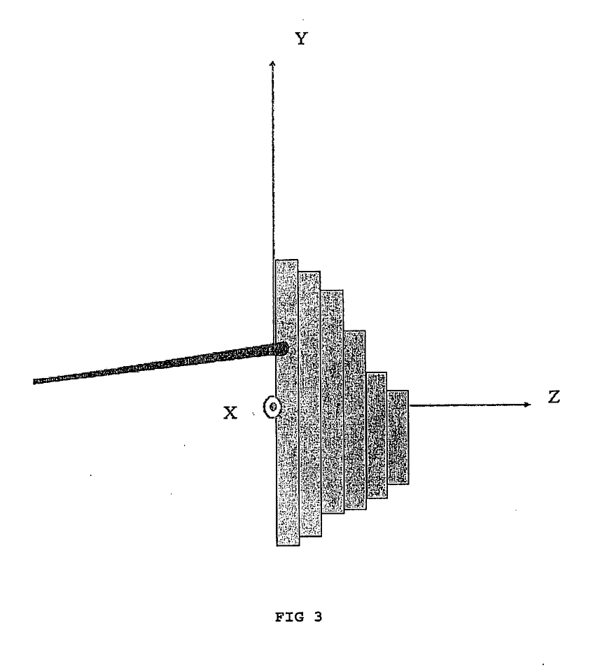

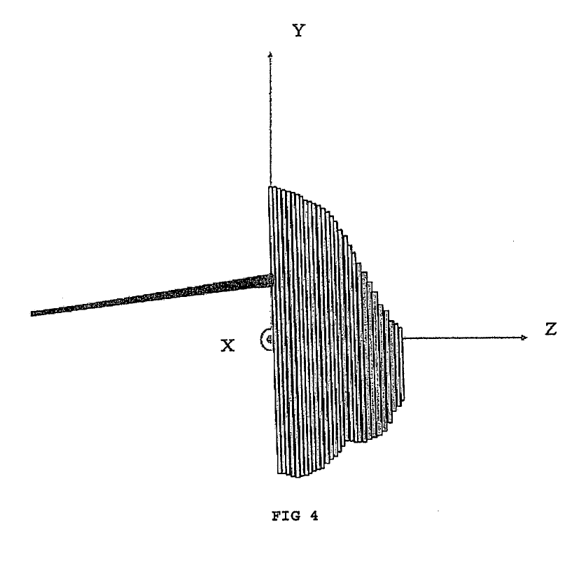

In a homogeneous material, instead of layers that would be orthogonal to the Z axis, one particular implementation of this method would result in very thin layers that are slightly tilted compared to the plane orthogonal to the Z axis, as can be seen from FIGS. 3 and 4. Other implementations are equally envisaged.

During the irradiation, the energy of the beam should be verified with a checking device such as multilayer Faraday cups in order to verify that the beam has the expected energy.

It is thus observed, in a particularly advantageous manner, that the method and the device according to the present invention do not use elements such as collimators, compensators, diffusers or path modulators, which makes the implementation of said method significantly less cumbersome.

In addition, it is observed that, according to the present invention, no movement of the patient is involved. The irradiation procedure resulting therefrom will be less cumbersome, faster and more accurate. Therefore, it will also be less expensive. Better conformation of the dose delivered to the target volume will thus be obtained, and in a minimum amount of time.

According to one particularly advantageous characteristic, it is observed that the movement of the spot in the 3D irradiation volume takes place without interruption of the beam, which allows a considerable saving in time and reduces the risk of sub-dosing between two consecutive irradiation points.

As it is sometimes desirable to irradiate the volume several times (=repainting), the device is capable of optimizing the speed at which the energy of the incident beam is modified to the amount of dose to be deposited and to the number of times that the volume needs to be repainted. In case of high dose and many repainting, the evolution of the energy of the charged particle beam will be very slow. In case of small dose and few repaintings, the speed of evolution of the energy can be significantly increased.

According to the methodology used, it is envisaged to cover the volume target several times in order to limit the dose delivered point by point during each passage, which increases the safety while at the same time limiting the problems due to the movements of the organs inside the body, for instance the breathing.

Preferably, the dose delivered during each passage represents about 2% of the total dose to be delivered for each voxel or irradiation volume.

In addition, the safety is also increased in this manner. The reason for this is that any problem associated with an imprecision of one of the two parameters will be automatically corrected by the other.

The methodology used consists in determining the dose corresponding to each spot by predefining the intensity of the beam and the scanning speed for each irradiation volume (or voxel), with the aid of a planning and processing computer device. During the irradiation, dose cards are permanently set up with the aid of measurements carried out by detection devices such as ionization chambers 3, 8 and other diagnostic elements. The 3D position of the spot and possibly the intensity of the proton or light ion beam are modified in real time and preferably simultaneously.

By envisaging to simultaneously vary the 3D coordinates of the spot, together with the scanning speed of the spot and the intensity of the proton or light ion beam, it is possible to obtain an adjustment of the dose to be delivered for each volume element with increased flexibility.

The intensity of the beam and the scanning speed, as well as the X, Y and Z position of the spot will be instantaneously recalculated and readjusted so as to ensure that the prescribed dose is effectively delivered to the target volume.

1. A method for treating or irradiating a target volume with a charged particle beam, the target volume being associated three coordinates according to a X, Y and Z direction, the Z coordinate corresponding to the beam direction while the X and Y coordinates correspond to directions perpendicular to the Z direction, said charged particle beam producing in said target volume an irradiation spot, the method comprising:
continuously scanning said spot in the X and Y directions; and
applying a continuous movement to said spot in the Z direction by seamlessly modifying an energy of said beam, wherein applying a continuous movement in Z direction and continuous scanning in X and Y direction are performed simultaneously and are effective for performing a continuous 3D scanning of the target volume.

2. The method for treating or irradiating a target volume according to claim 1, further comprising:
measuring and checking continuously a position of the spot in the target volume; and
adapting and correcting the position of said spot through a control loop for real time correction.

3. The method for treating or irradiating a target volume according to claim 1 or 2, further comprising modifying a scanning speed of the spot according to the 3D directions (X, Y, Z) while the target volume is being irradiated.

4. The method for treating or irradiating a target volume according to claim 3, further comprising modifying the beam intensity while the target volume is being irradiated.

5. The method for treating or irradiating a target volume according to claim 4, wherein said modifications are determined by an algorithm for planning.

6. A device for irradiating a target volume with a charged particle beam, wherein said charged particle beam generates an irradiating spot located within the target volume and to which are associated three coordinates according to a X, Y and Z direction, the Z coordinate corresponding to the beam direction while the X and Y coordinates correspond to directions in a plane perpendicular to the Z direction, the device comprising:
a scanning device configured to deflect the particle beam along the X and Y directions;
an energy variation device configured to continuously vary an energy of the beam; and
a central controlling device effective to control continuously the scanning device and the energy variation device by a planning and control algorithm effective for obtaining a conformation of the irradiation dose to the target volume.

7. The device according to claim 6, wherein the scanning device is arranged for moving the spot continuously in both X and Y directions.

8. The device according to claim 6 or 7, wherein the energy variation device is arranged for seamlessly varying the energy of the beam, thereby moving the spot continuously in Z direction.

9. The device according to claim 8, wherein the central controlling device allows a volume element within the target volume to be irradiated several times by the spot.

10. The device according to claim 6, further comprising a particle accelerator configured to produce the charged particle beam.

11. The device according to claim 10, wherein the energy variation device for the particle beam are located immediately after an extraction of the charged particle beam from the accelerator.

12. The device according to claim 11, wherein the energy variation device for the particle beam comprises an energy degrader, an energy absorber, or an energy selection device.

13. The device according to claim 6, further comprising an irradiation head and wherein the scanning device comprises a scanning magnet for each of the X and Y directions and wherein the scanning magnets are located in the irradiation head.

14. The device according to claim 6, further comprising a beam intensity variation device configured to seamlessly vary an intensity of the charged particle beam.

15. The device according to claim 14, wherein the central controlling device is adapted to carry out the control of the scanning device, of the energy variation device and of the beam intensity variation device with a planning and control algorithm, the algorithm including a control loop effective for correcting in real time trajectories of the spot.

16. The device according to claim 6, further comprising a computer system or sequencer implementing a treatment algorithm allowing a determination of an irradiation dose corresponding to each irradiation volume or voxel by predetermining the beam intensity, the beam energy and the scan speed in the X, Y and Z directions for each irradiation volume or voxel.

17. The device according to claim 16, wherein the scanning means, the energy-variation device and the beam intensity variation device act by moving the spot with no interruption of the beam.

18. The device according to claim 6, further comprising at least one detection device selected from the group consisting of an ionization chamber, a diagnostic element and combination thereof, the detection device effective for allowing measurements to be performed so as to check a conformation of the irradiation dose to the target volume.

19. A software program for being run on a computer and arranged for generating controlling commands to the scanning device and to the energy variation device of a device according to claim 6, the controlling commands effective for obtaining continuous movement of an irradiation spot.

20. (canceled)
